(19) United States
(12) Patent Application Publication
(10) Pub. No.: US 2019/0302484 A1
(43) Pub. Date: Oct. 3, 2019

(54) SMART GLASSES AND WEARING INSTRUCTION METHOD FOR SMART GLASSES

(71) Applicant: BOE TECHNOLOGY GROUP CO., LTD., Beijing (CN)

(72) Inventors: Xiao ZHANG, Beijing (CN); Lu TONG, Beijing (CN); Dacheng ZHANG, Beijing (CN); Shuo CHEN, Beijing (CN)

(21) Appl. No.: 16/254,875

(22) Filed: Jan. 23, 2019

(30) Foreign Application Priority Data
Apr. 3, 2018 (CN) 201810291259.4

(51) Int. Cl. G02C 11/00; G06F 1/16; G01L 1/20; A61B 90/50

(52) U.S. Cl. CPC G02C 11/10; G06F 1/163; G06K 9/00255; A61B 90/50; G01L 1/205

(57) ABSTRACT

The present disclosure relates to the technical field of glasses, and provides smart glasses. The smart glasses include a sensor unit configured to acquire wearing information of a wearer; an information inquiry unit, configured to acquire reference wearing information of the wearer; a comparison unit, configured to compare the wearing information with the reference wearing information; and an instruction unit, configured to send instruction information when a difference between the wearing information and the reference wearing information exceeds a threshold.

### CROSS REFERENCE

The present application claims the priority of Chinese Patent Application No. 201810291259.4, and filed on Apr. 3, 2018, and the entire contents thereof are incorporated herein by reference to serve as a portion of the present application.

### TECHNICAL FIELD

The present disclosure relates to the technical field of glasses, and in particular, to smart glasses and a wearing instruction method for smart glasses.

### BACKGROUND

When an object seen by two eyes are imaged on a retina, impressions on the left and right sides are combined to generate a stereoscopic impression, and a 3D picture with a stereoscopic depth is formed in the brain. Based on this principle, a 3D display makes two human eyes to respectively see a left-eye picture and a right-eye picture of an image, to generate a 3D stereoscopic effect. 3D camera and display technology has more applications in medical diagnosis and surgery field, mainly in that the 3D surgery technology based on 3D camera and 3D display can assist surgeons to obtain spatial layering feeling about internal tissues, to clearly distinguish tissue surfaces and blood vessels and to accurately position surgical points. The 3D surgery technology can effectively prevent large bleeding for patients, and can reduce the surgery operation time.

The above information disclosed in this Background section is only used to enhance an understanding of the background of the present disclosure, and thus it may include information that does not constitute the prior art known to those of ordinary skill in the art.

### SUMMARY

The present disclosure provides smart glasses and a wearing instruction method for the smart glasses.

The additional aspects and advantages of the present disclosure will be set forth in part in the following description, and will in part become apparent from the description and learned from practice of the present disclosure.

According to one aspect of the present disclosure, there is provided smart glasses, including:

a sensor unit configured to acquire wearing information of a wearer;

an information inquiry unit, configured to acquire reference wearing information of the wearer;

a comparison unit, configured to compare the wearing information with the reference wearing information; and

an instruction unit, configured to send instruction information when a difference between the wearing information and the reference wearing information exceeds a threshold.

In an exemplary embodiment of the present disclosure, the wearing information includes: identity information of the wearer, model information of the smart glasses, and matching information between the wearer and the smart glasses; and the reference wearing information includes: reference identity information of the wearer, reference model information of the smart glasses, and reference matching information between the wearer and the smart glasses.

In an exemplary embodiment of the present disclosure, the sensor unit includes:

a camera configured to acquire the identity information of the wearer; and

a plurality of pressure sensors disposed at positions where the wearer is in contact with the smart glasses, and configured to acquire the matching information between the wearer and the smart glasses.

In an exemplary embodiment of the present disclosure, the instruction unit is provided in multiple, and the multiple instruction units are disposed in one-to-one correspondence with the pressure sensors.

In an exemplary embodiment of the present disclosure, the plurality of pressure sensors includes:

at least two first pressure sensors disposed on a side of a nose pad of the smart glasses that is in contact with the wearer; and

at least two second pressure sensors disposed on a side of an earpiece of the smart glasses that is in contact with the wearer.

In an exemplary embodiment of the present disclosure, the smart glasses further include:

a confirmation button configured for the wearer to press when the wearer first wears the smart glasses and confirms that the wearing is appropriate, so as to store the wearing information acquired by the sensor unit to generate the reference wearing information.

In an exemplary embodiment of the present disclosure, the confirmation button is provided in at least two.

In an exemplary embodiment of the present disclosure, the smart glasses further include:

a communication unit, configured to transmit the wearing information and the instruction information; and

a storage unit, configured to store the reference wearing information.

In an exemplary embodiment of the present disclosure, the smart glasses are 3D glasses.

According to one aspect of the present disclosure, there is provided a wearing instruction method for smart glasses, including:

acquiring wearing information of a wearer;

acquiring reference wearing information of the wearer;

comparing the wearing information with the reference wearing information; and

sending instruction information when a difference between the wearing information and the reference wearing information exceeds a threshold.

In an exemplary embodiment of the present disclosure, the wearing information includes: identity information of the wearer, model information of the smart glasses, and a plurality of pieces of pressure information between the wearer and the smart glasses; and the reference wearing information includes: reference identity information of the wearer, reference model information of the smart glasses, and a plurality of pieces of reference pressure information between the wearer and the smart glasses.

In an exemplary embodiment of the present disclosure, after acquiring the wearing information of the wearer, the wearing instruction method further includes:

determining whether it is the first time wearing for the wearer;

when it is the first time wearing, storing the wearing information to generate the reference wearing information when the wearer confirms that the wearing is appropriate.

It should be understood that both the foregoing general description and the following detailed description are exemplary and explanatory only and are not restrictive of the disclosure.

### BRIEF DESCRIPTION OF THE DRAWINGS

The above and other features and advantages of the present disclosure will become more apparent from the detailed description of the exemplary embodiments with reference to the accompanying drawings.

FIG. 1 is a schematic structural diagram of smart glasses according to an embodiment of the present disclosure;

FIG. 2 is a schematic diagram of a frame structure of smart glasses according to an embodiment of the present disclosure;

FIG. 3 is a schematic diagram showing a working process of the smart glasses of the present disclosure;

FIG. 4 is a schematic flow chart of a wearing instruction method for smart glasses according to the present disclosure.

### DETAILED DESCRIPTION

Exemplary embodiments will now be described more fully with reference to the accompanying drawings. However, the exemplary embodiments can be embodied in a variety of forms and should not be construed as being limited to the embodiments set forth herein. Rather, these embodiments are provided to make the present disclosure more complete and thorough, and to fully convey the concept of the exemplary embodiments to those skilled in the art. The same reference numerals in the drawings denote the same or similar structures, and thus their detailed description will be omitted.

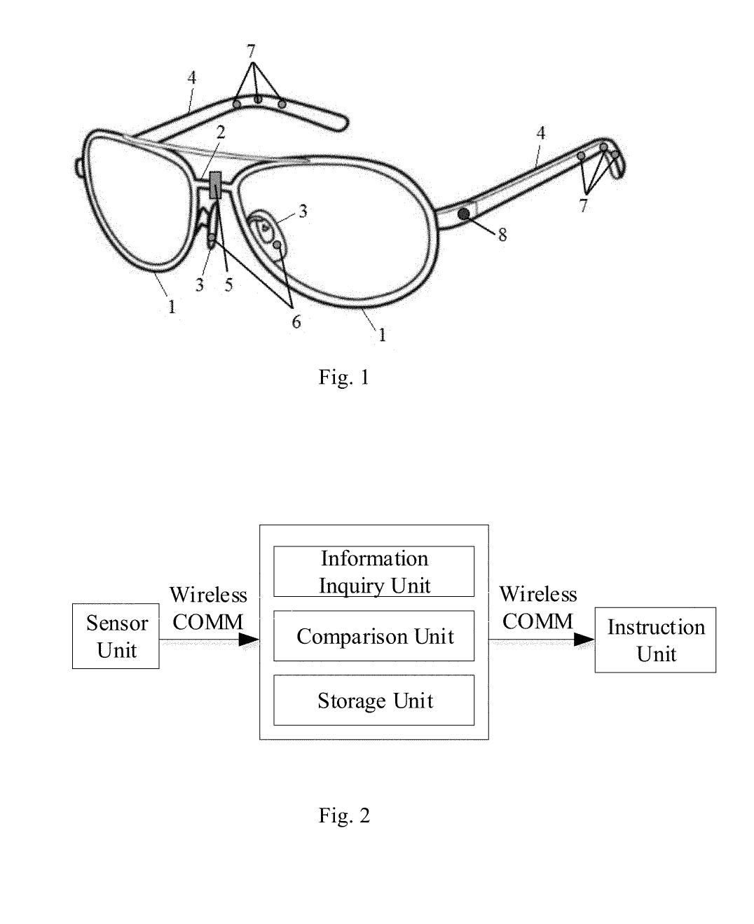

Referring to the schematic structural diagram of an embodiment of the smart glasses shown in FIG. 1; the frame structure of the smart glasses is the same as that of ordinary glasses. Specifically, the smart glasses may include two frames 1 and a connecting rod 2 for connecting the two frames 1, two nose pads 3 provided respectively on the two frames 1, and two earpieces 4 provided respectively on the two frames 1.

Referring to FIG. 2, which is a schematic diagram of a frame structure of the smart glasses according to an embodiment. The smart glasses may further include a sensor unit, an information inquiry unit, a comparison unit, an instruction unit, and other components. The sensor unit may be configured to acquire wearing information of a wearer. The information inquiry unit may be configured to obtain reference wearing information of the wearer. The comparison unit may be configured to compare the wearing information with the reference wearing information. The instruction unit may be configured to send instruction information when a difference between the wearing information and the reference wearing information exceeds a threshold.

In this exemplary embodiment, the wearing information may include: identity information of the wearer, model information of the smart glasses, and matching information between the wearer and the smart glasses. The reference wearing information may include: reference identity information of the wearer, reference model information of the smart glasses, and reference matching information between the wearer and the smart glasses.

The identity information is information that can determine the identity of the wearer that is unique to make him apart from others. For example, the identity information may include the wearer's name, age, ID number, work department, face image, and the like. The model information of the smart glasses is mainly determined according to the appearance of the glasses, and the glasses of different appearances have different models. That is, the glasses of the same appearance have the same model, and the wearing manners of glasses of different shapes are different. The matching information may be pressure information between the wearer and the smart glasses, and may also be other information such as distance information.

The smart glasses may also include a confirmation button 8 that can be configured for the wearer to press when the wearer wears the smart glasses for the first time (in other words, the wearer is new) and confirms that the wearing is appropriate, so as to store wearing information acquired by the sensor unit to generate reference wearing information. In the exemplary embodiment, two confirmation buttons 8 are respectively disposed on either side of one end of the two earpieces 4 near the frames 1, with one confirmation button being disposed on one earpiece 4. When both of the two confirmation buttons 8 are pressed simultaneously, it may indicate confirming that the wearing is appropriate. Providing two confirmation buttons 8 can prevent the wearer from accidentally pressing the buttons. Before the wearer wears a certain model of smart glasses for the first time, the identity information of the wearer is acquired by the camera 5. After the wearer wears the smart glasses, and the wearer confirms that the wearing is appropriate, the wearer presses the confirm buttons 8. The pressure sensor acquires a pressure value between the wearer and the smart glasses, and the memory stores the identity information of the wearer, the model information of the glasses, and the pressure value between the wearer and the smart glasses to generate a reference identity information of the wearer, a reference model information of the smart glasses, and the reference matching information between the wearer and the smart glasses (i.e. the reference pressure value).

In the exemplary embodiment, the sensor unit may include a camera 5 and a plurality of pressure sensors and the like. The camera 5 may be configured to acquire the identity information of the wearer. The camera 5 can acquire the identity information of the wearer by collecting the face image of the wearer; and can also obtain the identity information of the wearer's name, age, ID number, work department, etc. by collecting the badge worn by the wearer. However, as two-dimensional codes are used frequently these days, the camera 5 can also acquire the identity information of the wearer by collecting the wearer's two-dimensional code, and can also acquire the identity information of the wearer by collecting the wearer's fingerprint information. The camera 5 is installed on the connecting rod 2 of the smart glasses.

A plurality of pressure sensors are provided at positions where the wearer is in contact with the smart glasses. In the exemplary embodiment, eight pressure sensors are provided, two of which are first pressure sensors 6, and six of which are second pressure sensors 7. Three second pressure sensors 7 form a group, and there are totally two groups. The two groups are disposed symmetrically on the two earpieces 4. The first pressure sensor 6 is provided on the side of each of the two nose pads 3 of the smart glasses, which is in contact with the wearer. The second pressure sensor 7 is disposed on a side of the earpiece 4 of the smart glasses which is in contact with the wearer. One of the second pressure sensor 7 is disposed at the bending point of the earpiece 4, and the other two second pressure sensors 7 are disposed on both sides of the pressure sensor. In addition, in other exemplary embodiments of the present disclosure, the pressure sensor may be provided in six, seven, ten or more, as long as it is disposed at a position in contact with the wearer.

A plurality of pressure sensors may be configured to obtain the matching information between the wearer and the smart glasses. When the wearer wears the smart glasses, the plurality of pressure sensors may contact the wearer to sense the pressure between the wearer and the smart glasses, and the pressure value is the matching information. However, when a distance sensor is installed, the obtained matching information is a distance value.

The information inquiry unit may be configured to obtain reference wearing information of the wearer. In the exemplary embodiment, whether it is the first time wearing may be determined by whether there is identity information of the wearer and matching model information of the smart glasses in the memory. That is, if there is identity information of the wearer and matching model information of the smart glasses, it is not the first time wearing, and if there is no identity information of the wearer and matching model information of the smart glasses, it is the first time wearing. After it is determined that it is not the first time wearing, the information inquiry unit searches for the reference matching information between the wearer and the smart glasses according to the identity information of the wearer and the model information of the smart glasses to be worn. However, the information inquiry unit may also search for the reference wearing information stored in the memory one by one.

According to the present disclosure, if there is no identity information of the wearer and matching model information of the smart glasses, the wearer is considered to be “first time wearing” or “new”, however this does not necessarily mean that the wearer has never worn the mart glasses before. Also, the “first time wearing” should not be interpreted as limiting the number of times the smart glasses are worn.

The comparison unit may be configured to compare the wearing information with the reference wearing information. In the exemplary embodiment, after the information inquiry unit acquires the reference matching information (i.e., the reference pressure value) between the wearer and the smart glasses according to the identity information of the wearer and the model information of the smart glasses to be worn, the comparison unit compares the pressure value acquired by each sensor with corresponding reference pressure value.

According to the present disclosure, the units and modules such as the information inquiry unit and the comparison unit may include an application specific integrated circuit (ASIC), an electronic circuit, a processor (e.g., a shared processor, a proprietary processor, or a group processor, etc.) for executing one or more software or firmware programs, and a memory, a merge logic circuit, and/or other suitable components that support the functions described. However, the present disclosure is not limited thereto, and the units and modules may also be formed by software modules stored on a storage medium or a combination of software modules and hardware modules.

When the difference between the wearing information and the reference wearing information exceeds a threshold, the instruction unit sends instruction information. The instruction unit is provided in multiple, and the multiple instruction units are in one-to-one correspondence with the pressure sensor. In an exemplary embodiment, the instruction units may be LED lights, and the instruction units may also be provided in eight, and each of them is disposed in a position corresponding to one pressure sensor and can be seen by human eyes. For example, at the position on the left nose pad 3 that can be seen by the human eye there is provided an instruction unit corresponding to the pressure sensor on the left nose pad 3, which can facilitate the assistant to learn about which position is not worn properly and needs adjustment.

After the comparison unit performs comparison, if the difference between the pressure value acquired by one sensor and the reference pressure value exceeds a first threshold value, the LED light corresponding to the sensor is turned on constantly to send instruction information. When the difference between the pressure value acquired by the sensor and the reference pressure value exceeds a second threshold, the LED light corresponding to the sensor flashes to send instruction information. The first threshold is less than the second threshold. Therefore, when the LED light is turned on constantly to send instruction information, it means that the smart glasses need to be slightly adjusted, and when the LED light flashes to send instruction information, it means that the smart glasses need to be adjusted significantly. For example, when the difference between the pressure value measured by the sensor on the left nose pad 3 and the reference pressure value corresponding to the left nose pad 3 as stored in the memory exceeds the first threshold, the LED light corresponding to the sensor on the left nose pad 3 is turned on constantly to send instruction information. When the difference between the pressure value measured by the sensor on the left nose pad 3 and the reference pressure value corresponding to the left nose pad 3 as stored in the memory exceeds the second threshold, the LED light corresponding to the sensor on the left nose pad 3 flashes to send instruction information.

However, the instruction unit may also be a voice instruction unit, and the voice instruction unit can be set in one. When the difference between the pressure value measured by the pressure sensor at one position and the corresponding reference pressure value exceeds a threshold value, the voice instruction unit can directly report the position through voice. For example, when the difference between the pressure value measured by the sensor on the left nose pad 3 and the reference pressure value corresponding to the left nose pad 3 as stored in the memory exceeds the threshold value, the voice instruction unit may directly send an instruction voice “left nose pad 3”, to instruct the assistant that the wearing is not appropriate at the position of the left nose pad 3 and needs to be adjusted. The present disclosure intents to include other obvious modifications or substitutions that are not specifically listed therein.

The smart glasses may further include a communication unit and a storage unit. The communication unit may be configured to transmit the wearing information and the instruction information. Although the present disclosure is not limited thereto, the communication unit may include a network interface. The communication unit may be a wireless communication unit. The storage unit may be configured to store the reference wearing information. The storage unit may include either or both of volatile memories and non-volatile memories. The information inquiry unit, the comparison unit, and the storage unit may be installed on the smart glasses, and in this case, the communication unit is not needed. Alternatively, instead of being installed on the smart glasses, the information inquiry unit, the comparison unit, and the storage unit may be implemented in a general server to function as the information inquiry unit, the comparison unit, and the storage unit, and in this case, the communication unit is necessary.

In the exemplary embodiment, the smart glasses are 3D glasses. However, it can also be myopia glasses, hypermetropia glasses, astigmatism glasses, and the like.

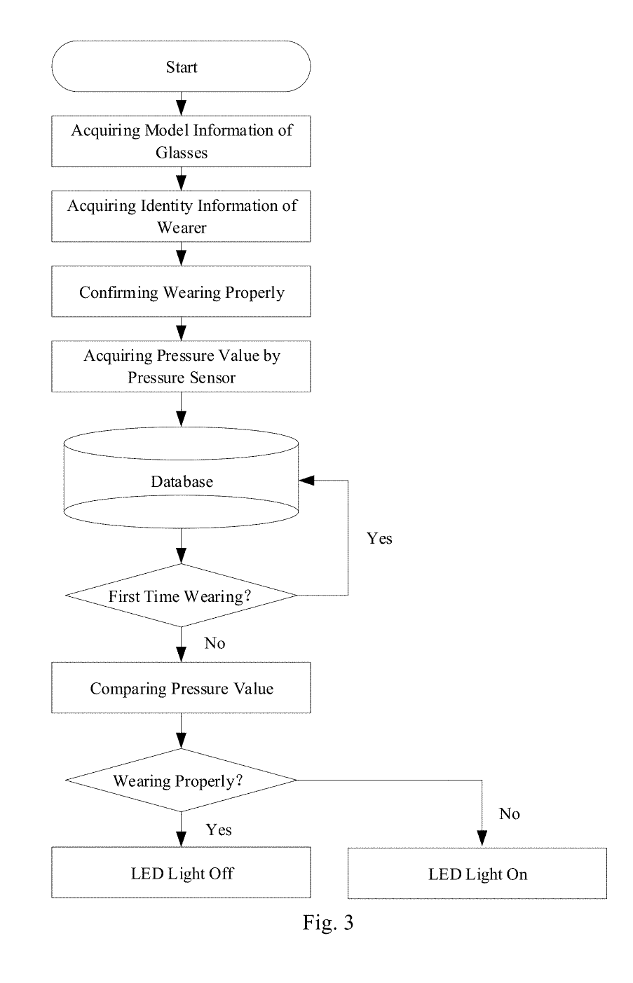

With reference to the workflow diagram shown in FIG. 3, the usage of the smart glasses will be described in detail as follows.

At the beginning of usage, the wearer turns on the switch of the smart glasses. The power unit supplies power to the smart glasses, and each unit module starts to work normally.

Acquisition of the identity information of the first-time wearer. Identity information needs to be acquired for each wearer when the wearer uses the smart glasses for the first time. Firstly, the face image, the badge image, the fingerprint image or the two-dimensional code of the wearer are acquired through the camera 5, and the image information together with the model information of the glasses are transmitted to the memory through the wireless communication unit. The memory records the identity information of the wearer and the model information of the glasses. When the wearer feels that the glasses are properly worn, the wearer simultaneously presses two confirmation buttons 8. After the confirmation on wearing, the pressure sensor starts to acquire the pressure, and the pressure values acquired by the pressure sensors in different positions are different. The wireless communication unit transmits the pressure values of the respective pressure sensors to the memory, and the memory stores the pressure values of the respective pressure sensors in association with the identity information of the wearer and the model information of the glasses to generate reference wearing information. The memory stores data related to each model of smart glasses worn by each wearer to form a database.

Wearing detection and instruction. When the wearer uses the smart glasses, the camera 5 on the smart glasses firstly acquires the identity information of the wearer, and then the pressure sensor on the smart glasses acquires data. The identity information, the model information of the smart glasses, and the pressure information are transmitted to the information inquiry unit through the wireless communication unit. The information inquiry unit matches the identity information and the model information with the reference identity information and the reference model information in the database. If the reference identity information and the reference model information matching the identity information and the model information are found in the database, it may be determined that the it is not the first time wearing, and no reference identity information and no reference model information matching the identity information and the model information are found in the database, it may be determined that it is the first time wearing. If it is the first time wearing, the above mentioned first time acquisition of identity information of the wearer is performed to update the database. If it is not the first time wearing, the information inquiry unit further search for the reference matching information (i.e., the reference pressure information) when the wearer wears the model of smart glasses. Then, the comparison unit compares the pressure value acquired by the pressure sensor with the reference pressure value. If the difference between the pressure values acquired by each pressure sensor and the corresponding reference pressure value is within the threshold range, it may be considered that the glasses are worn properly by the wearer, and the wearer may use the glasses in normal. If the difference between the pressure value acquired by one of the pressure sensors and the corresponding reference pressure value exceeds the first threshold, the LED light corresponding to the pressure sensor is turned on constantly to send instruction information. When the difference between the pressure value acquired by the pressure sensor and the reference pressure value exceeds the second threshold, the LED light corresponding to the pressure sensor flashes to send instruction information. The first threshold is less than the second threshold. Therefore, when the LED light is turned on constantly to send instruction information, it means that the smart glasses need to be slightly adjusted, and when the LED light flashes to send instruction information, it means that the smart glasses need to be adjusted significantly.

When the assistant sees that the LED light is turned on, the assistant may assist the doctor to adjust the smart glasses. During the adjustment process, the pressure sensor also performs real-time tracking detection until the difference between the pressure value detected by the pressure sensor and the reference pressure value in the database is within the threshold range. The adjustment of the glasses is completed and the glasses are worn properly.

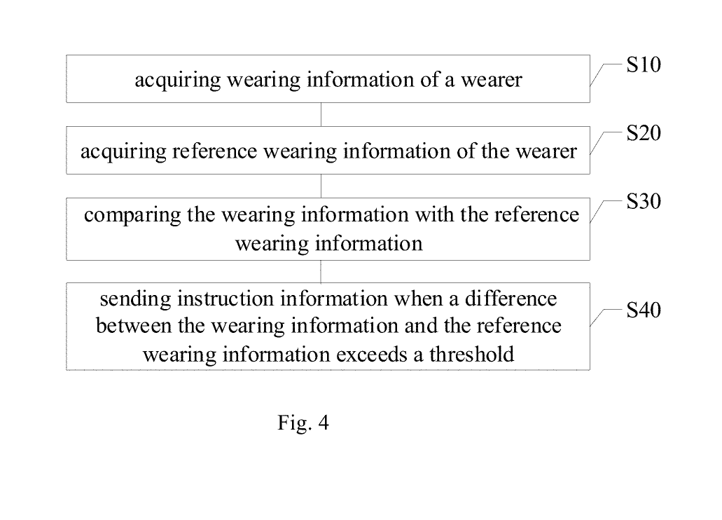

Further, corresponding to the smart glasses, the present disclosure further provides a wearing instruction method for the smart glasses. As shown in FIG. 4, which is a flowchart of the wearing instruction method for the smart glasses, the instruction method includes the following steps.

In Step S10, wearing information of a wearer is acquired.

In Step S20, reference wearing information of the wearer is acquired.

In Step S30, the wearing information is compared with reference wearing information.

In step S40, instruction information is sent when a difference between the wearing information and the reference wearing information exceeds a threshold.

In the exemplary embodiment, the wearing information includes: identity information of the wearer, model information of the smart glasses, and a plurality of pieces of pressure information between the wearer and the smart glasses. The reference wearing information includes: reference identity information of the wearer, reference model information of the smart glasses, and a plurality of pieces of reference pressure information between the wearer and the smart glasses.

In the exemplary embodiment, after acquiring the wearing information of the wearer, the wearing instruction method further includes: determining whether it is the first time wearing for the wearer; if it is the first time wearing, when the wearer confirms that the wearing is appropriate, the wearing information is stored to generate the reference wearing information.

The wearing instruction method has been described in detail in the above description of the smart glasses, details of which will not be repeated herein.

As can be seen from the above technical solutions, the present disclosure has at least one of the following advantages and beneficial effects.

In the smart glasses of the present disclosure, the wearing information of the wearer is acquired through the sensor unit; the reference wearing information of the wearer is acquired through the information query unit; then, the comparing unit compares the wearing information with the reference wearing information; finally, when the difference between the wearing information and the reference wearing information exceeds the threshold, the instruction unit sends instruction information. On one hand, the comparison unit can determine that the wearer is not wearing the glasses properly, and the instruction unit can instruct the assistant to assist in adjusting the glasses, and the doctor does not need to instruct the assistant to assist in adjusting the glasses. On the other hand, the degree of intelligence is high and will not affect the doctor's surgery operation.

The features, structures, or characteristics described above may be combined in any suitable manner in one or more embodiments, and the features discussed in the various embodiments are interchangeable, if possible. In the description above, numerous specific details are set forth to provide a thorough understanding. However, one skilled in the art will appreciate that the technical solution of the present disclosure may be practiced without one or more of the specific details, or other methods, components, materials, and the like may be employed. In other instances, well-known structures, materials or operations are not shown or described in detail to avoid obscuring aspects of the present disclosure.

In the present specification, the terms “a”, “an”, “the”, “said” and “at least one” are used to indicate the presence of one or more elements/components/etc. The terms “comprising”, “including” and “having” are used to indicate the meaning of non-exclusive inclusion and there may be further elements/components/etc. in addition to the listed elements/components/etc. The terms “first”, “second” and “third” etc. are used only as references, rather than limitations on the number of objects.

It should be understood that the present disclosure does not limit its application to the detailed structure and arrangement of the components proposed in the present specification. The present disclosure may have other embodiments, and may be implemented and carried out in various manners. The foregoing variations and modifications are within the scope of the present disclosure. It is to be understood that the present disclosure disclosed and claimed herein extends to all alternative combinations of two or more individual features that are mentioned or apparent in the drawings. All of these different combinations constitute a number of alternative aspects of the present disclosure. The embodiments described in the specification are illustrative of the best mode for carrying out the present disclosure and will enable those skilled in the art to utilize the present disclosure.

What is claimed is:

1. Smart glasses, comprising:
a sensor unit, configured to acquire wearing information of a wearer;
an information inquiry unit, configured to acquire reference wearing information of the wearer;
a comparison unit, configured to compare the wearing information with the reference wearing information; and
an instruction unit, configured to send instruction information in a case where a difference between the wearing information and the reference wearing information exceeds a threshold.

2. The smart glasses according to claim 1, wherein
the wearing information comprises: identity information of the wearer, model information of the smart glasses, and matching information between the wearer and the smart glasses; and
the reference wearing information comprises: reference identity information of the wearer, reference model information of the smart glasses, and reference matching information between the wearer and the smart glasses.

3. The smart glasses according to claim 2, wherein the sensor unit comprises:
a camera, configured to acquire the identity information of the wearer; and
a plurality of pressure sensors, disposed at positions where the wearer is in contact with the smart glasses, and configured to acquire the matching information between the wearer and the smart glasses.

4. The smart glasses according to claim 3, wherein the instruction unit is provided in multiple, and the multiple instruction units are disposed in one-to-one correspondence with the pressure sensors.

5. The smart eyewear according to claim 3, wherein the plurality of pressure sensors comprises:
at least two first pressure sensors, disposed on a side of at least one nose pad of the smart glasses that is in contact with the wearer; and
at least two second pressure sensors, disposed on a side of at least one earpiece of the smart glasses that is in contact with the wearer.

6. The smart glasses according to claim 1, further comprising:
a confirmation button, configured for the wearer to press in a case where the wearer is new and confirms that the wearing is appropriate, so as to store the wearing information acquired by the sensor unit to generate the reference wearing information.

7. The smart glasses according to claim 6, wherein the confirmation button is provided in at least two.

8. The smart glasses according to claim 1, further comprising:
a communication unit, configured to transmit the wearing information and the instruction information; and
a storage unit, configured to store the reference wearing information.

9. The smart glasses according to claim 1, wherein the smart glasses are 3D glasses.

10. A wearing instruction method for smart glasses, comprising:
acquiring wearing information of a wearer;
acquiring reference wearing information of the wearer;
comparing the wearing information with the reference wearing information; and
sending instruction information in a case where a difference between the wearing information and the reference wearing information exceeds a threshold.

11. The wearing instruction method for smart glasses according to claim 10, wherein the wearing information comprises: identity information of the wearer, model information of the smart glasses, and a plurality of pieces of pressure information between the wearer and the smart glasses; and the reference wearing information comprises: reference identity information of the wearer, reference model information of the smart glasses, and a plurality of pieces of reference pressure information between the wearer and the smart glasses.

12. The wearing instruction method for smart glasses according to claim 10, wherein after acquiring the wearing information of the wearer, the wearing instruction method further comprises:
determining whether the wearer is new;
in a case where it is determined that the wearer is new, storing the wearing information to generate the reference wearing information when the wearer confirms that the wearing is appropriate.

13. The wearing instruction method for smart glasses according to claim 10, wherein the step of determining whether the wearer is new comprises:
determining that the wearer is new in a case where identity information of the wearer, model information of the smart glasses matching the identity information of the wearer are not stored in a memory of the smart glasses.
